(19) United States
(12) Patent Application Publication
(10) Pub. No.: US 2011/0171286 A1
(43) Pub. Date: Jul. 14, 2011

(54) HYALURONIC ACID COMPOSITIONS FOR DERMATOLOGICAL USE

(75) Inventors: GOUSSE CECILE, Pringy Cedex (FR); Pierre F. LEBRETON, Pringy Cedex (FR); Nicolas PROST, Pringy Cedex (FR)

(73) Assignees: ALLERGAN, INC., Irvine, CA (US); ALLERGAN Industrie, SAS, Pringy (FR)

(21) Appl. No.: 12/687,048

(22) Filed: Jan. 13, 2010

(51) Int. Cl. A61K 8/73; A61K 8/60; A61K 8/14; A61Q 19/00; A61Q 19/08

(52) U.S. Cl. 424/450; 514/54; 514/27

(57) ABSTRACT

The disclosure provides hyaluronic acid (HA) gel formulations and methods for treating the appearance of the skin. The formulations hyaluronic acid and at least one additional constituent selected from the group consisting of vitamin B, C and vitamin E, wherein the formulation exhibits greater stability than an HA gel formulation without the additional constituent. Methods for treating lines, wrinkles, fibroblast depletions, and scars with the disclosed composition are provided as well.

### BACKGROUND

Skin aging is a progressive and irreversible phenomenon. Aging of the skin occurs over time and is impacted by lifestyle factors, such as alcohol consumption, tobacco and sun exposure.

Aging of the facial skin can be characterized by atrophy, slackening, and fattening. Atrophy corresponds to a massive reduction of the thickness of skin tissue. Slackening of the subcutaneous tissues leads to an excess of skin and ptosis and leads to the appearance of drooping cheeks and eye lids. Fattening refers to an increase in excess weight by swelling of the bottom of the face and neck. These changes are typically associated with dryness, loss of elasticity, and rough texture.

Vitamin C and hyaluronic acid (HA) are known to have an effect on skin. Vitamin C is the L-enantiomer of ascorbate and has a well-described role in collagen development. Vitamin C is involved in the hydroxylation of collagen, which allows it to assume its triple-helix structure. Vitamin C is also known for its antioxidant effects and is well tolerated.

HA is a natural polysaccharide. It is a polymer of disaccharides that are themselves composed of D-glucuronic acid and N-acetylglucosamine, linked to one another by alternating beta-1,4 and beta-1,3 glycosidic linkages. The polymers of this recurring unit may be from 10² and 10⁴ kilo Daltons (kDa) in size, in vivo. Hyaluronic acid represents a natural constituent of the dermis, where it plays an important role in the hydration and elasticity of skin. There is a strong correlation between the water content in the skin and levels of HA in the dermal tissue. As skin ages, the amount and quality of HA in the skin is reduced. These changes lead to drying and wrinkling of the skin.

The use of HA in cosmetic and dermatological applications is known. HA is tolerated well and there is no immunogenicity associated with its use. The low incidence of side effects has lead to the use of HA for the treatment of wrinkles, fine lines, and scars. HA is subject to degradation through different pathways (e.g. enzymatic, temperature, free radicals), and therefore, its longevity in vivo is limited.

There are numerous disclosures of HA, vitamin C, and C-glycosides in the art, including: U.S. Pat. No. 6,921,819 (a process for cross-linking solid hyaluronic acid (HA) by reacting it with a polyfunctional linker during hydration of the HA); U.S. Pat. No. 6,685,963 (acrylic particles of HA); U.S. Publication No. 2006/0194758 (a method for making a hydrogel by cross linking high and low molecular weight sodium HAs); U.S. Publication No. 2009/0036403 (cross-linking HA with a tetra functional PEG epoxide to provide “tunably” cross-linked HA); U.S. Publication No. 2009/0143331 (a HA dermal filler with a degradation inhibitor, such as chondroitin sulphate, in order to provide a longer lasting filler); U.S. Publication No. 2009/0143348 (HA combined with a steroid); and U.S. Publication No. 2009/0155314 (HA combined with a botulinum toxin). Additionally, U.S. Publication Nos. 2009/0148527, 2009/0093755, and 2009/0022808 disclose HA in microspheres, cross-linked with collagen, and coated with a protein, respectively. Further disclosures of HA include: WO 2009/034559 (a process for aesthetic and/or reparative treatment of the skin with compositions that contain at least one C-glycoside derivative); WO 2009/024719 (cosmetic and pharmaceutical compositions that contain HA and a C-glycoside derivative useful for filling recesses/depressions in the skin, restore volume of the body or the face, and to reduce the sign of aging); WO 2007/128923 (a method for preparing a biocompatible gel with controlled release of one or more active lipophilic and/or amphiphilic ingredients); U.S. Patent Publication No. 2009/0018102 (compositions containing HA and at least one retinoid or salt/derivative thereof in combination with an oligosaccharide and a HA degradation inhibitor, to treat wrinkles, lines fibroblast depletions and scars); U.S. Pat. No. 3,763,009 (a process for improving the oxidation resistance of ascorbic acid by subjecting a mixture of ascorbic acid, maltose and/or oligosaccharides to an enzyme derived from genera Aspergillus, Penicillium or others to enzymatically convert the mixture into ascorbic acid glucoside); U.S. Pat. No. 5,616,611 (a α-Glycosyl-L-ascorbic acid that exhibits no direct reducing activity, is stable, and is useful as a stabilizer, quality-improving agent, antioxidant, physiologically active agent, a UV-absorbant in pharmaceutical and cosmetic industries); U.S. Pat. No. 5,843,907 (the production and use of a crystalline 2-O-α-D-glucopyranosyl-L-ascorbic acid suitable for vitamin C enriching agents, food stuffs, pharmaceuticals, and cosmetics); and EP 0539196 (an industrial scale preparation of high purity 2-O-α-D-glucopyranosyl-L-ascorbic acid) and US 2002/0151711. Commercial products incorporating HA and/or vitamin C agents include: MESOGLOW® products, REVITACARE®, and NCTF® 135/135HA Mesotherapy products. Each of the above-cited references and printed publications are individually incorporated herein by reference in their entirety.

### BRIEF SUMMARY

Described herein are compositions and methods useful for treating the skin.

In one embodiment, the disclosure provides a dermal filler formulation comprising hyaluronic acid (HA) and at least one additional constituent selected from the group consisting of vitamin C and vitamin E, wherein the formulation exhibits greater stability than an HA gel formulation without the additional constituent.

In another embodiment, a method is provided for treating fine lines, wrinkles, fibroblast depletions, or scars afflicting a subject comprising administering to the subject an effective amount of a formulation comprising HA and at least one additional constituent selected from the group consisting of vitamin C and vitamin E, wherein the formulation exhibits greater stability than an HA gel formulation without the additional constituent, and wherein the appearance of the fine lines, wrinkles, fibroblast depletions, or scars is diminished.

The disclosure further provides a dermal filler comprising about 1 to about 60 mg/mL HA and an additional constituent selected from the group consisting of ascorbyl-2-glucoside and TPGS, wherein the dermal filler exhibits greater stability than a dermal filler comprising HA without the additional constituent.

### DESCRIPTION OF THE DRAWINGS

FIG. 1 is a representation of the structure of an ascorbyl-2-glucoside, also known as AA2G™ (Hayashibara Co., Japan).

FIG. 2 is a graph showing the synthesis of pro-collagen (% control) for control, gel+lidocaine 0.3%, AA2G™ 0.6% in phosphate buffer, and gel+AA2G™ 0.6%+lidocaine 0.3%.

FIG. 3 is a graph showing the extrusion force over time (3 yr equivalent at 25° C.) in compositions: control, AA2G™ plus lidocaine, and AA2G™ plus lidocaine and TPGS.

FIG. 4 is a graph showing the pH over time (3 yr equivalent at 25° C.) in compositions: control, AA2G™ plus lidocaine, and AA2G™ plus lidocaine and TPGS.

FIG. 5 is a graph of tan delta 1 Hz over time (3 yr equivalent at 25° C.) in compositions: control, AA2G™ plus lidocaine, and AA2G™ plus lidocaine and TPGS.

FIG. 6 is an HPLC analysis (C18 column, eluent: sodium phosphate buffer (pH=2.2)/2-propanol 10%, 0.7 ml/min; detection at 260 nm) of AA2G™, lidocaine, and IPA (coeluent) after autoclaving (3 yr equivalent at 25° C.).

FIG. 7 is a graph comparing antioxidant properties in compositions: control versus JUVEDERM® Ultra plus AA2G™, and JUVEDERM® plus lidocaine.

### DETAILED DESCRIPTION

The disclosure relates to formulations and methods for treating the skin. The disclosure provides a dermal filler formulation comprising hyaluronic acid (HA) and at least one additional constituent selected from the group consisting of vitamin C and vitamin E, wherein the formulation exhibits greater stability than an HA gel formulation without the additional constituent.

The presence of an additional constituent provides a stability and longevity that is not exhibited in formulations containing HA without the additional constituent. The disclosed formulations are homogenous, uncolored, clear, cohesive gels that are stable to heat and oxidation. The HA in the formulation is stable to heat and oxidation. The additional constituent is hydrophilic and provides protection to the HA from degradation. Without wishing to be bound by any particular theory, the incorporation of an additional constituent to a HA composition may inhibit free-radical scavenging at the injection/implant site, thereby prolonging implant duration.

The disclosure further provides a method of treating fine lines, wrinkles, fibroblast depletions, and/or scars afflicting a subject. The method comprises administering to the subject an effective amount of a formulation comprising hyaluronic acid and at least one additional constituent selected from the group consisting of vitamin C and vitamin E, wherein the formulation exhibits greater stability than a HA gel formulation without the additional constituent.

The subject can be any mammal, preferably a human of any age, gender or race. Although typically a subject experiencing the signs of aging skin is an adult, subjects experiencing premature aging or other skin conditions suitable for treatment (for example, a scar) with the HA gel formulation can be treated as well.

HA, as used herein, can be cross-linked or not cross-linked. Although any pharmaceutically or cosmetically acceptable HA can be used in the disclosed compositions and formulations, in certain embodiments, the HA utilized includes those sold as JUVEDERM®, JUVEDERM® 30, JUVEDERM® Ultra Plus, JUVEDERM® Ultra injectable gel. Additional HAs include NTCF® 135HA (Filorga, Paris),

One method for achieving a HA composition that persists is to encapsulate the additional constituent(s) within the HA polymer network itself or into vessels (Le., liposomes, micelles, and/or polymerized vesicles) within the network that enable local (injection site), sustained and controlled release of the constituents. This method allows the constituent to avoid the natural degradation mechanisms encountered in vivo. In one embodiment of an encapsulation method, a constant supply of a constituent is supplied to the polymer network over a period of weeks or months. The additional constituent can be incorporated into the polymer network by adsorption or by an encapsulation process. In the latter case, the additional constituent is allowed to mix with the HA network at a highly hydrated state, followed by dehydration of the network to control the release kinetics (e.g. final swelling ratio of the polymer). A highly hydrated state corresponds to an HA concentration that is less than about 20 mg/mL. The final swelling ratio can be controlled by adjusting the pH or partially dehydrating the HA network.

In certain embodiments, the formulation comprises a HA gel matrix and an additional constituent. HA is a known hydrogel. The gel can be injectable, bioresorbable, monophasic, or biphasic. In some embodiments, the additional constituent can be directly incorporated into the HA gel. In other embodiments, in order to increase affinity with the medium or increase stability, modification of the molecule by derivatization or encapsulation of the constituent can be performed, as described above. For instance, certain oily molecules cannot be introduced directly into a hydrophilic matrix, and lead to a heterogeneous product. Derivatization of the molecule by grafting hydrophilic moieties is required to increase homogeneity of the gel. In some embodiments, the gel composition can include a biocompatible or biodegradable vessel. Such vessels can be composed of non-covalently or covalently linked self-assembled molecules such as liposomes, micelles, and polymerized vesicles.

A liposome is a vesicle composed of one or more bilayer membranes formed of naturally-derived phospholipids with mixed lipid chains (such as egg phosphatidylethanolamine), or of pure surfactant components like dioleoylphosphatidylethanolamine (DOPE). Liposomes, usually but not by definition, contain a core of aqueous solution; lipid structures that contain no aqueous material are called micelles. A micelle is an aggregate of surfactant molecules dispersed in a liquid colloid. A typical micelle in aqueous solution forms an aliphatic aggregate with the hydrophilic “head” regions in contact with surrounding solvent, sequestering the hydrophobic “tail” regions in the micelle center. This type of micelle is known as a normal phase micelle (oil-in-water micelle). Inverse micelles have the head groups at the center with the tails extending out (water-in-oil micelle). Micelles are often approximately spherical in shape, however, other forms, including shapes such as ellipsoids, cylinders, and bilayers are also possible. The shape and size of a micelle is a function of the molecular geometry of its surfactant molecules and solution conditions such as surfactant concentration, temperature, pH, and ionic strength. The process of forming micelles is known as micellisation and forms part of the phase behavior of many lipids according to their polymorphism.

The HA gel can be made by any known, suitable methods. Cross-linked HA gels typically have high viscosity and require considerable force to extrude through a fine needle. Uncross-linked HA is often used as a lubricant to facilitate the extrusion process. However, especially in HA dermal fillers and implants, uncross-linked HA does not contribute to the persistence of the final product in vivo. In fact, the more cross-linked HA that is replaced by uncross-linked HA to tune the rheological properties of the dermal filler (for a fixed total HA concentration), the lower the degradation resistance of the product will be. Instead, according to the disclosed formulation, a cross-linked HA is utilized and an additional constituent can be used both to extend the longevity and affect the rheological properties of the final product. Accordingly, the formulations disclosed herein require less extrusion force for administration compared to formulations containing cross-linked HA without the additional constituent. Further, the formulations exhibit increased stability compared to formulations containing HA without the additional constituent. Stability is determined by assessing the homogeneity, color, and clarity, pH, and rheological properties of the gel formulation. The formulations disclosed herein are considered stable if they remain homogenous, colorless, and/or clear, and exhibit stable pH and rheology. The disclosed formulations remain stable for at least about 6 months, at least about 1 year, at least about 2 years or at least about 3 years. In certain embodiments, the formulations are stable for at least about 4 years, or at least about 5 years.

A cross-linking agent can be used to cross-link the HA according to the present disclosure. The cross-linking agent may be any agent known to be suitable for cross-linking HA and its derivatives via hydroxyl groups. Suitable cross-linking agents include but are not limited to, 1,4-butanediol diglycidyl ether, 1,4-bis(2,3-epoxypropoxy)butane, and/or 1,4-bisglycidyloxybutane (commonly known as BDDE), 1,2-bis(2,3-epoxypropoxy)ethylene, and 1-(2,3-epoxypropyl)-2,3-epoxycyclohexane. The use of more than one cross-linking agent or a different cross-linking agent is included from the scope of the present disclosure.

Dermal fillers can be used to treat moderate to severe facial wrinkles and folds such as nasolabial folds (those lines that extend from the nose to the corners of the mouth). In one embodiment, a dermal filler can be a gel implant formulation that includes HA and an additional constituent. The formulations disclosed herein can further include additional cosmetic agents that supplement and improve the appearance of skin. The cosmetic active ingredients may include, but are not limited to, antioxidants, vitamins, tension agents, and moisturizers.

The formulations disclosed herein can be injected with a syringe into the mid to deep dermis of the face. The dermis is the subsurface skin layer that contains connective tissue, nerve endings, and blood vessels. The formulations, when administered as dermal fillers can improve skin appearance by lifting and adding volume to the wrinkles and folds in the treatment area. Further, in certain embodiments, improvement can be seen due to increased collagen production that results from administration of the formulation.

As used herein, “cosmetic” is an adjective referring to improving the appearance of a surface or covering defects. Typically, cosmetic compositions can be used to improve aesthetic rather than functional aspects of a surface. Most commonly, cosmetic compositions are formulated for application as a health and beauty treatment or for affecting personal appearance of the body, for example, keratinous surfaces such as skin, hair, nails, and the like.

As used herein, “formulation” and “composition” may be used interchangeably and refer to a combination of elements that is presented together for a given purpose. Such terms are well known to those of ordinary skill in the art.

As used herein, “carrier,” “inert carrier,” and “acceptable carrier” may be used interchangeably and refer to a carrier which may be combined with the presently disclosed HA gel in order to provide a desired composition. Those of ordinary skill in the art will recognize a number of carriers that are well known for making specific remedial pharmaceutical and/or cosmetic compositions. Desirably, the carrier is suitable for application to keratinous surfaces or other areas of the body. Upon application, cosmetically acceptable carriers are substantially free of adverse reactions with skin and other keratinous surfaces. For example, the cosmetic carriers may take the form of fatty or non-fatty creams, milky suspensions or emulsion-in-oil or oil-in-water types, lotions, gels or jellies, colloidal or non-colloidal aqueous or oily solutions, pastes, aerosols, soluble tablets or sticks.

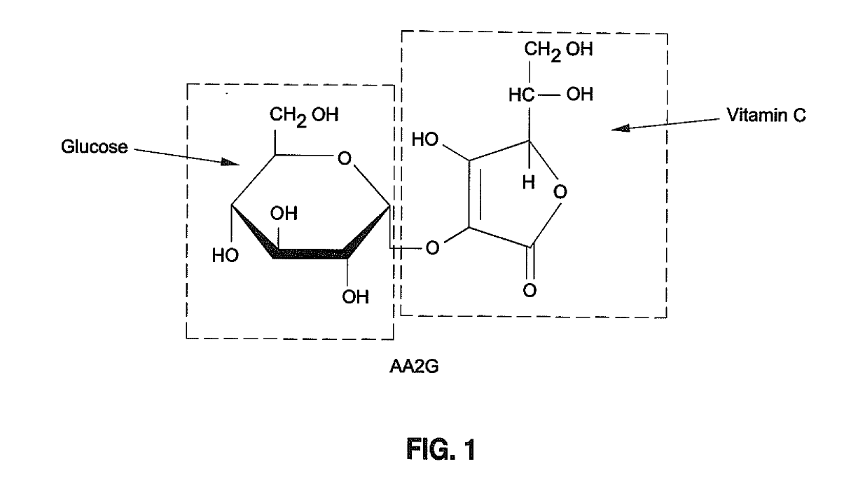

Examples of additional agents which can be included in the present pharmaceutical or cosmetic formulations are anti-itch, anti-cellulite, anti-scarring, and anti-inflammatory agents, anesthetics, anti-irritants, vasoconstrictors, vasodilators, as well as agents to prevent/stop bleeding, and improve/remove pigmentation, moisturizers, desquamating agents, tensioning agents, anti-acne agents. Anti-itch agents can include methyl sulphonyl methane, sodium bicarbonate, calamine, allantoin, kaolin, peppermint, tea tree oil and combinations thereof. Anti-cellulite agents can include forskolin, xanthine compounds such as, but not limited to, caffeine, theophylline, theobromine, and aminophylline, and combinations thereof. Anesthetic agents can include lidocaine, benzocaine, butamben, dibucaine, oxybuprocaine, pramoxine, proparacaine, proxymetacaine, tetracaine, and combinations thereof. Anti-scarring agents can include IFN-.gamma., fluorouracil, poly(lactic-co-glycolic acid), methylated polyethylene glycol, polylactic acid, polyethylene glycol and combinations thereof. Anti-inflammatory agents can include dexamethasone, prednisolone, corticosterone, budesonide, estrogen, sulfasalazine, mesalamine and derivatives and combinations thereof. Additionally, active agents such as epinephrine, thymidine, cytidine, uridine, antiypyrin, aminocaproic acid, tranexamic acid, eucalyptol, allantoin, glycerin, and sodium selenite, can be included. Formulations can further comprise degradation inhibitors. Degradation inhibitors, include but are not limited to, glycosaminoglycans (e.g., heparin, heparin sulfate, dermatan sulfate, chrondroitin sulfate, o-sulfated HA, Inamarin, and amygdalin), antioxidants (e.g. ascorbic acid, melatonin, vitamin C, vitamin E), proteins (e.g., serum hyaluronidase inhibitor), and fatty acids (e.g. saturated C₁₀ to C₂₂ fatty acids). In certain embodiments, additional active agent is an antioxidant. In certain embodiments, the antioxidant comprises a vitamin C such as ascorbyl-2-glucoside (available as AA2G™, Hayashibara Co., Japan) (FIG. 1), and/or a vitamin E such as d-alpha-tocopheryl polyethylene glycol 1000 succinate (TPGS).

In certain embodiments, the additional active agents that can be included in the present pharmaceutical or cosmetic formulations are anti-itch, anti-scarring, and anti-inflammatory agents, anesthetics, anti-irritants, vasoconstrictors, as well as agents to prevent/stop bleeding, and improve/remove pigmentation, moisturizers, tensioning agents, anti-acne agents. Anti-itch agents can include methyl sulphonyl methane, sodium bicarbonate, calamine, allantoin, kaolin, peppermint, tea tree oil, camphor, menthol, hydrocortisone. Anesthetic agents can include lidocaine, benzocaine, butamben, dibucaine, oxybuprocaine, pramoxine, proparacaine, proxymetacaine, tetracaine. Anti-scarring agents can include IFN-.gamma, fluorouracil, poly(lactic-co-glycolic acid), methylated polyethylene glycol, polylactic acid, polyethylene glycol and combinations thereof. Anti-inflammatory agents can include dexamethasone, prednisolone, corticosterone, budesonide, estrogen, sulfasalazine, mesalamine, cetirizine, diphenhydramine, antiprine, methyl salicylate, loratadine, and derivatives and combinations thereof. Anti-irritants can include thymol, bisabolol. Healing agents can include allantoin, eucalyptol, chitosane, cytidine, thimidine, uridine, lanoline. Anti-bleeding: epinephrine, norepinephrine, phenylephrine, synephrine, naphazoline, aminocaproic acid, tranexamic acid, ethamsylate, vitamin K. Collagen promoters can include retinol, peptide sequences. Additionally, active agents such as epinephrine, thymidine, cytidine, uridine, antiypyrin, aminocaproic acid, eucalyptol, sodium selenite, can be included. Formulations can further comprise degradation inhibitors such as glycosaminoglycans (e.g., heparin, heparin sulfate, dermatan sulfate, chrondroitin sulfate, o-sulfated HA, lnamarin, and amygdalin, glucosamine), antioxidants (e.g. ascorbic acid, melatonin, vitamin C, vitamin E, sodium selenite, glutathion, retinoic acid, coenzyme, beta-carotene, allopurinol, mannitol, caffeic acid, caffeine, polyphenol, theobromine, catechin), proteins (e.g., serum hyaluronidase inhibitor), and fatty acids (e.g. saturated C₁₀ to C₂₂ fatty acids); vitamin B and complex, and combinations thereof.

In some embodiments, the HA is present at a concentration of about 1 to about 60 mg/mL, or about 10 to about 40 mg/mL, or about 20 to about 30 mg/mL. In certain embodiments, the HA is present in a concentration of about 20 to about 25 mg/mL. In certain embodiments, the HA is present at a concentration of 24 mg/mL. The additional constituent can be present in an amount of about 0.001 to about 10% w/w, or from about 0.001 to about 5% w/w, or from 0.3 to about 3% w/w.

In certain embodiments, the disclosure provides a dermal filler comprising (a) about 90 wt %, or about 95 wt %, or about 100 wt % of a high molecular weight (about 1 million to about 3 million Daltons) HA; and (b) 0 wt %, or about 5 wt %, or about 10 wt % of a low molecular weight (less than 1 million Daltons) HA. In certain embodiments, the HA is present in the dermal filler at a concentration of about 10 to about 24 mg HA/mL dermal filler and the HA is about 4% to about 11% cross-linked. In certain embodiments, the cross linker is 4-butane diol diglycidyl ether (BDDE). The dermal filler can further comprise about 0.1 wt % or 0.6 wt %, or 1.0 wt % of an ascorbyl-2-glucoside, such as AA2G™ (Hayashibara, Japan). In a preferred embodiment, 0.6 wt % AA2G™ (i.e., 6mg AA2G™/g HA) is utilized and renders a concentration of 2.1012 mM AA2G™

Topical formulations of AA2G™ are known. However, there are no subdermally administered formulations of AA2G™ available, which is likely due to the fact that a topical AA2G™ is not thought to lend itself to an injectable formulation. The disclosure provides the first injectable formulation of AA2G™ that efficacious, compatible, and stable over time.

The disclosed compositions are also well suited for mesotherapy. Mesotherapy is a non-surgical cosmetic treatment technique involving intra-epidermal, intra-dermal, and/or subcutaneous injection of an agent (micronutrients, vitamins, mineral salts, etc). The compositions are administered in the form of small multiple droplets into the epidermis, dermo-epidermal junction, and/or the dermis.

The formulations of the disclosure can be injected utilizing needles with a diameter of about 0.26 to about 0.4 mm and a length ranging from about 4 to about 14 mm. Alternately, the needles can be 21 to 32 G and have a length of about 4 mm to about 70 mm. Preferably, the needle is a single-use needle. The needle can be combined with a syringe, catheter, and/or a pistol (for example, a hydropneumatic-compression pistol).

The formulations can be administered once or over several sessions with the subject spaced apart by a few days, or weeks. For instance, the subject can be administered a formulation every 1, 2, 3, 4, 5, 6, 7, days or every 1, 2, 3, or 4, weeks. The administration can be on a monthly or bi-monthly basis. Further, the formulation can be administered every 3, 6, 9, or 12 months.

A pharmaceutical or cosmetic composition can optionally include one or more agents such as, without limitation, emulsifying agents, wetting agents, sweetening or flavoring agents, tonicity adjusters, preservatives, buffers antioxidants and flavonoids. Tonicity adjustors useful in a pharmaceutical composition of the present disclosure include, but are not limited to, salts such as sodium acetate, sodium chloride, potassium chloride, mannitol or glycerin and other pharmaceutically acceptable tonicity adjusters. Preservatives useful in the pharmaceutical compositions described herein include, without limitation, benzalkonium chloride, chlorobutanol, thimerosal, phenyl mercuric acetate, and phenyl mercuric nitrate. Various buffers and means for adjusting pH can be used to prepare a pharmaceutical composition, including but not limited to, acetate buffers, citrate buffers, phosphate buffers and borate buffers. Similarly, antioxidants useful in pharmaceutical compositions are well known in the art and include for example, sodium metabisulfite, sodium thiosulfate, acetylcysteine, butylated hydroxyanisole and butylated hydroxytoluene. Flavonoids are compounds found in plants that are well known to have diverse beneficial biochemical and antioxidant effects. Subcategories of flavonoids include: flavones, flavonols, flavanonse and flavanonols. Examples of flavonoids include: luteolin, apigenin, tangeritin, quercetin, kaempferol, myricetin, fisetin, isorhamnetin, pachypodol, rhamnazin, hesperetin, naringenin, eriodictyol, homoeriodictyol, taxifolin, dihydroquercetin, dihydrokaempferol, tannic acid, tannis, condensed tannis, and hydrolysable tannis. It is understood that these and other substances known in the art can be included in a pharmaceutical or cosmetic composition disclosed herein.

The pH of the disclosed formulations can be about 5.0 to about 8.0, or about 6.5 to about 7.5. In certain embodiments, the pH of the formulation is about 7.0 to about 7.4 or about 7.1 to about 7.3.

The formulations described herein can be contained in a single vial with each constituent administered simultaneously (i.e., in a ready to use formulation) or the constituents can be contained in separate vials. When the HA and additional constituent are in separate vials, the HA and the additional constituent can be mixed prior to administration. Regardless of the packaging, the compositions should be capable of withstanding a sterilization process, including but not limited to, steam sterilization, filtration, microfiltration, gamma radiation, ETO light or a combination thereof. In certain embodiments, the formulations can be steam sterilized (autoclaved) with no degradation of the physical properties. Further, the formulations are stable at 45° C. for at least about 30 days, or at least about 60 days, or at least about 90 days with no degradation of physical properties.

### EXAMPLES

### Example 1

### Properties of Formulations of NaHA and Water Soluble Molecules Are Tested

The active ingredient was incorporated into a NaHA matrix and autoclaved. The properties of the gel, aspect (i.e., color/clarity/homogeneity) and extrusion force were analyzed after sterilization at 3 years equivalent at room temperature. Table 1 shows that all formulations were clear, homogenous, and uncolored at the 3-year mark. The extrusion forces after autoclaving and at 3 years equivalent at room temperature are shown as well.

In conclusion, the incorporation of the molecules has no impact on gel properties and ingredient structure.

TABLE 1

| | | | Extrusion | Extrusion |
|---|---|---|---|---|
| | | | force (N) | force (N) |
| | Content | | after | 3 years ~ |
| Ingredient | (%) | Aspect | autoclaving | room T° C. |
| Allantoin | 0.3 | Clear | PASSED | PASSED |
| | 0.5 | Homogeneous | PASSED | PASSED |
| Cytidine | 0.5 | uncolored | PASSED | PASSED |
| | 1 | | PASSED | PASSED |
| Thymidine | 0.5 | | PASSED | PASSED |
| | 1 | | PASSED | PASSED |
| Uridine | 0.5 | | PASSED | PASSED |
| | 1 | | PASSED | PASSED |
| Antipyrin | 0.5 | | PASSED | PASSED |
| | 1 | | PASSED | PASSED |
| Aminocaproic | 0.5 | | PASSED | PASSED |
| acid | 1 | | PASSED | PASSED |
| Tranexamic acid | 0.5 | | PASSED | PASSED |
| Eucalyptol | 0.5 | | PASSED | PASSED |
| Sodium selenite | 0.1 | | PASSED | PASSED |
| Glycerin | 0.5 | | PASSED | PASSED |

Acceptance criteria: “Passed” means that the change of extrusion force (ΔF) was less than two Newtons (<2 N). In other words the measured ΔF of the extrusion force of the HA gel with the specified ingredients minus the extrusion force of the HA gel without the added ingredients was <2 N.

### Example 2

### Preparation of NaHA Gel Containing Vitamin C

Ascorbic acid (1% w/w) was incorporated into a NaHA matrix. (JUVEDERM® FORMA). The pH was adjusted to about 7 and composition was autoclaved. The gel obtained was clear, yellow and degraded.

### Example 3

### Alternative Preparation of NAHA Gel Containing Vitamin C

Magnesium Ascorbyl Phosphate (MAP) (0.6%, 1 or 2% w/w) was incorporated in a NaHA matrix (JUVEDERM® Ultra). The pH was adjusted to about 7 and the compositions were autoclaved. All gels obtained were uncolored and clear. The gel properties after autoclaving are shown in Table 2.

Extrusion force acceptance criteria: Conform with NaHA matrix specifications

TABLE 2

| | After autoclaving |
|---|---|
| Formulation | Extrusion force (N) |
| JUVEDERM ® Ultra + 0.6% MAP | PASSED |
| JUVEDERM ® Ultra + 1% MAP | PASSED |
| JUVEDERM ® Ultra + 2% MAP | PASSED |

Rheology data of the gel containing 2% MAP after autoclaving is shown in Table 3. Rheological properties are followed as a function of time using a controlled stress rheometer according to the following method: frequency sweep from 0.05 to 10 Hz with 0.8% controlled strain. A degradation of the gel was observed by rheology. TAN δ×HZ is a rheological characterisation which shows the ratio of viscous modulus to elastic modulus. It shows the degradation of the gel.

Δ Tan δ 1 Hz=(Tan δ 1 Hz formulation)−(Tan δ 1 Hz NaHA matrix)

Acceptance criteria: −0.1<Δ Tan δ 1 Hz<0.1

TABLE 3

| Formulation | Δ Tan δ 1 Hz |
|---|---|
| JUVEDERM ® Ultra + 2% MAP | 0.344 |

### Example 4

### Alternative Preparation of NAHA Gel Containing Vitamin C

Sodium Ascorbyl Phosphate (SAP) (0.6%, 1% and 2% w/w) was incorporated in an NaHA matrix (JUVEDERM® Ultra). The pH was adjusted to about 7 and composition was autoclaved. All gels obtained were uncolored and clear. The gel properties after autoclaving are shown in Table 4.

TABLE 4

| | After autoclaving |
|---|---|
| Formulation | Extrusion force (N) |
| JUVEDERM ® Ultra + 0.6% SAP | PASSED |
| JUVEDERM ® Ultra + 1% SAP | PASSED |
| JUVEDERM ® Ultra + 2% SAP | PASSED |

Rheology data of the gel containing 2% SAP after autoclaving is shown in Table 5. No degradation of the gel was observed by rheology.

TABLE 5

| Formulation | Δ Tan δ 1 Hz |
|---|---|
| JUVEDERM ® Ultra + 2% SAP | 0.089 |

### Example 5

### Alternative Preparation of NaHA Gel Containing Vitamin C

Ascorbic acid 2-Glucoside (AA2G™) at a concentration of 0.6%, 1% and 2% w/w was incorporated in an NaHA matrix (JUVEDERM® Ultra Plus). The pH was adjusted to about 7 and the composition was autoclaved. All gels obtained were uncolored and clear. The gel properties after autoclaving are shown in Table 6.

TABLE 6

| | After autoclaving |
|---|---|
| Formulation | Extrusion force (N) |
| JUVEDERM ® Ultra Plus + 0.6% AA-2G | PASSED |
| JUVEDERM ® Ultra Plus + 1% AA-2G | PASSED |
| JUVEDERM ® Ultra Plus + 2% AA-2G | PASSED |

The gels containing 0.6%, 1% and 2% were stable (pH, injection force) after autoclaving. Rheology data of the gels containing 0.6%, 1% and 2% w/w AA2G™ after autoclaving is shown in Table 7. No degradation of the gel was observed by rheology at each AA2G™ concentration.

TABLE 7

| Formulation | Δ Tan δ 1 Hz |
|---|---|
| JUVEDERM ® Ultra Plus + 0.6% AA2G ™ | −0.010 |
| JUVEDERM ® Ultra Plus + 1% AA2G ™ | −0.014 |
| JUVEDERM ® Ultra Plus + 2% AA2G ™ | −0.016 |

Rheological studies showed an slightly increase of the stability of the gel in the presence of the additive.

### Example 6

### Effect of Vitamin C on Aspect and Stability of the Gel

The shelf-life at 45° C. during 32 days was tested for the formulations prepared in example 5 and the NaHA matrix JUVEDERM® Ultra Plus. Rheology data of the gels containing 0.6%, 1% and 2% of AA2G™ are shown in Table 8.

TABLE 8

| Formulation | Δ Tan δ 1 Hz |
|---|---|
| JUVEDERM ® Ultra Plus + 0.3% AA2G ™ | −0.050 |
| JUVEDERM ® Ultra Plus + 0.5% AA2G ™ | −0.045 |
| JUVEDERM ® Ultra Plus + 1% AA2G ™ | −0.059 |

The gels containing ascorbyl glucoside maintained their properties after autoclaving and over a period of 32 days at 45° C. Rheological studies showed an increase of the stability of the gel in the presence of the additive.

### Example 7

### Preparation of NaHA Gel Containing Vitamin E

Tocopheryl Acetate (0.5% w/w) was incorporated into a NaHA matrix. (JUVEDERM® 30) and autoclaved. The gel obtained was unclear, white.

### Example 8

### Alternative Preparation of NaHA Gel Containing Vitamin E

Sodium Tocopheryl Phosphate (STP), at 0.4%, 1.2% w/w, was incorporated in a NaHA matrix (JUVEDERM® FORMA) and autoclaved. The gel obtained was unclear, white.

### Example 9

### Alternative Preparation of NaHA Gel Containing Vitamin E

Polyoxyethanyl-α-tocopheryl sebacate (0.7% w/w) was incorporated in a NaHA matrix (JUVEDERM® Ultra Plus) and autoclaved. The gel obtained was clear, but heterogenous.

### Example 10

### Alternative Preparation of NaHA Gel Containing Vitamin E

Tocopherol polyethylene glycol 1000 succinate (TPGS) was incorporated in varying concentrations (1%, 3.5% and 7% w/w) in a NAHA matrix (JUVEDERM® FORMA) and autoclaved. “JUVEDERM® FORMA” means the Juvederm formulation was used. All gels obtained were uncolored and clear. The gel properties after autoclaving are shown in Table 9.

TABLE 9

| Formulation | Extrusion force (N) |
|---|---|
| JUVEDERM ® FORMA + 1% TPGS | PASSED |
| JUVEDERM ® FORMA + 3.5% TPGS | PASSED |
| JUVEDERM ® FORMA + 7% TPGS | PASSED |

Rheology data of the gels containing 1%, 3.5% and 7% TPGS after autoclaving is shown in Table 10. No degradation of the gel was observed by rheology at each TPGS concentration.

TABLE 10

| Formulation | Δ Tan δ 1 Hz |
|---|---|
| JUVEDERM ® FORMA + 1% TPGS | 0.008 |
| JUVEDERM ® FORMA + 3.5% TPGS | −0.007 |
| JUVEDERM ® FORMA + 7% TPGS | −0.011 |

Rheological studies showed an slightly increase of the stability of the gel depending on the additive content.

### Example 11

### Stability of Formulations Containing Additional Ingredients

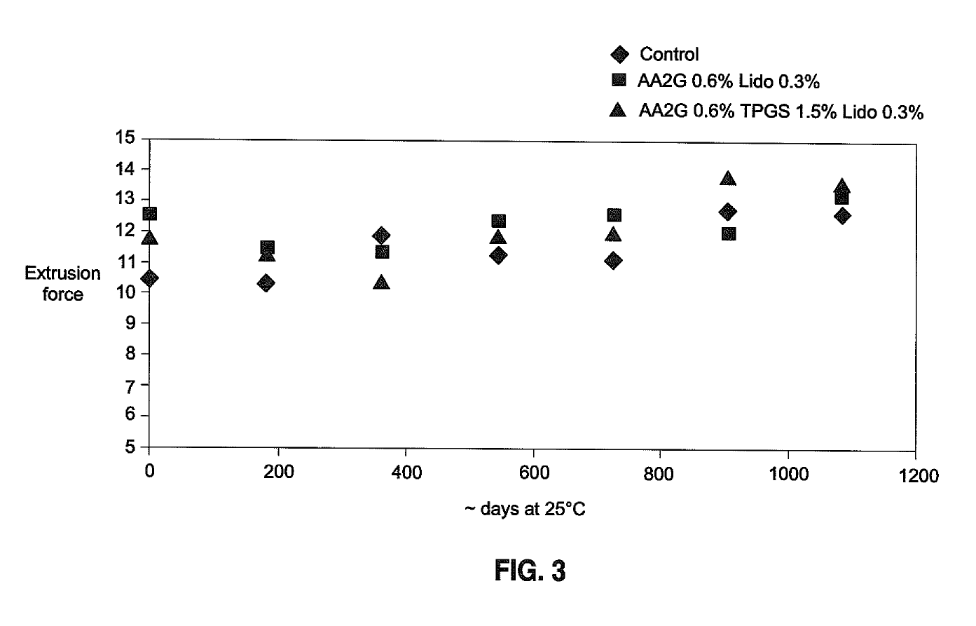

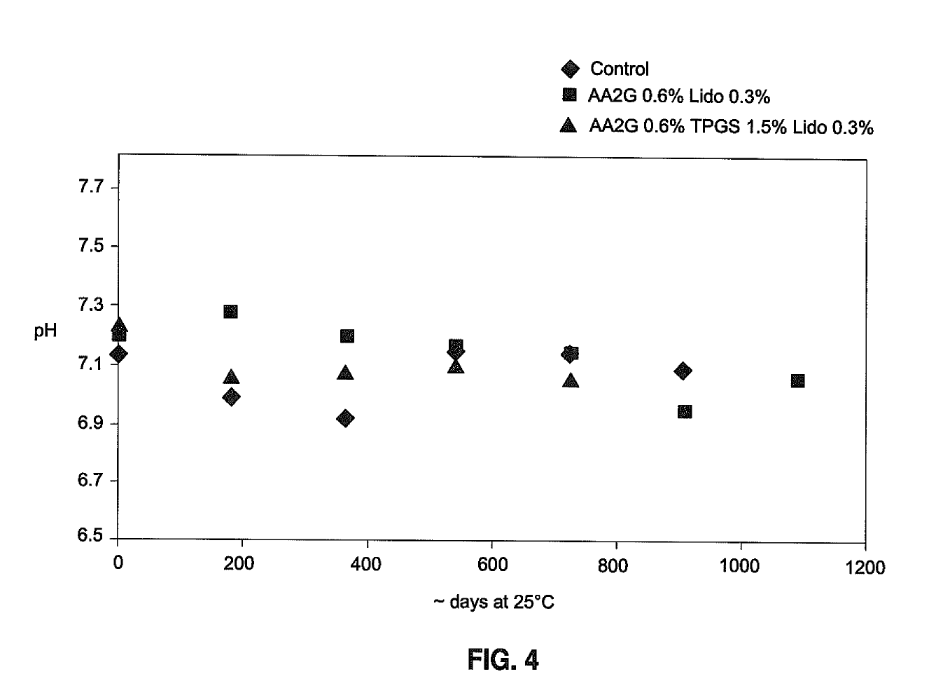

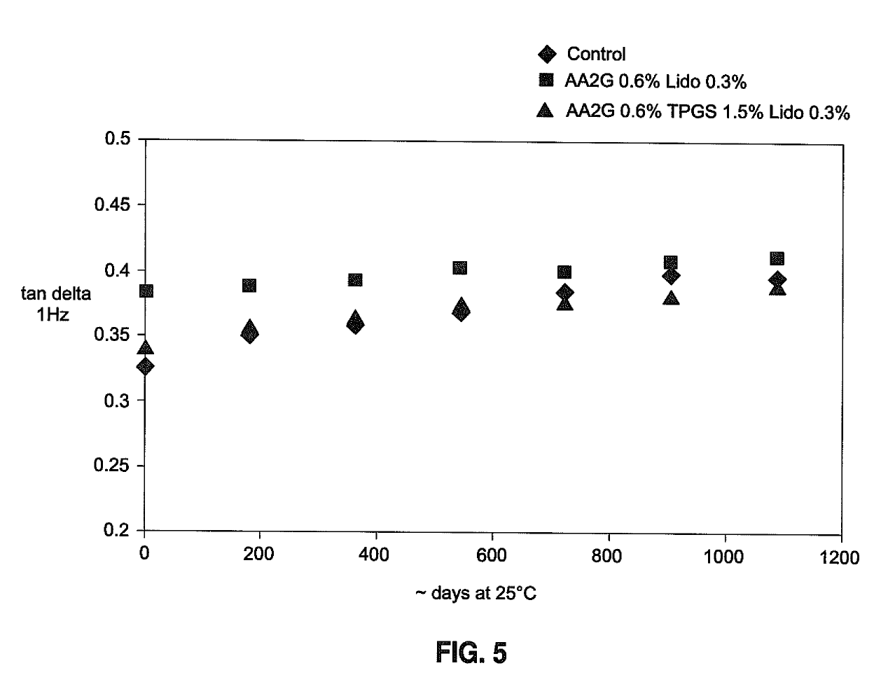

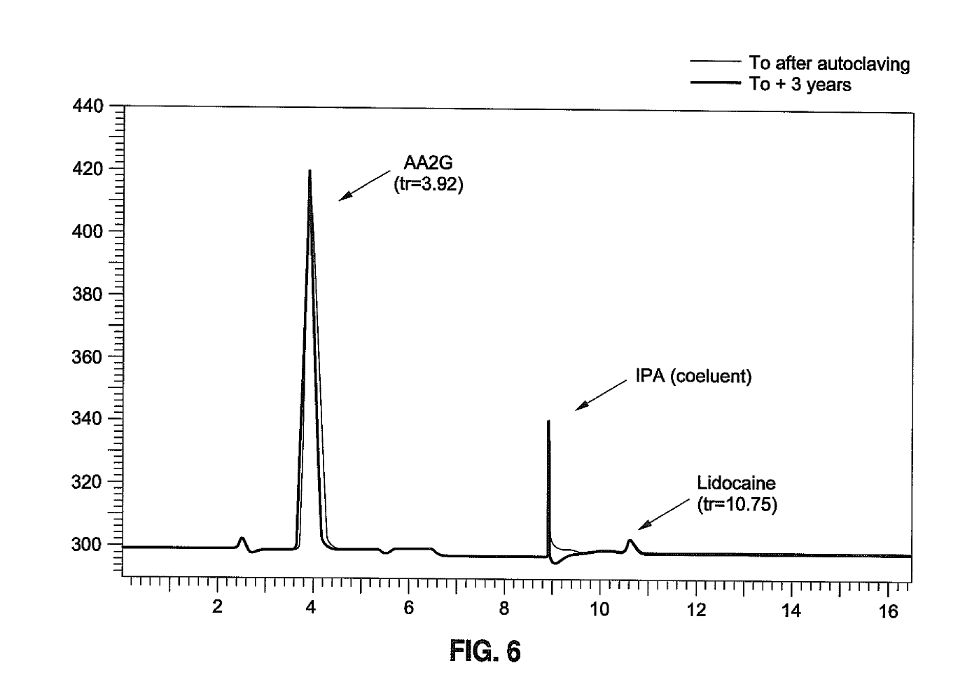

The stability of various formulations was tested. The ingredients shown in Table 11 were incorporated into a NaHA matrix, and autoclaved. The degradation of the formulations after autoclaving is shows in Table 11 and after 48 days at 45° C. in Table 12. The stability of extrusion force, pH, and degradation are shown over time in FIGS. 3, 4, and 5, respectively. HPLC analysis (C18 column; eluent: sodium phosphate buffer (pH 2.2), 2-propanol 10%, 0.7 ml/min; detection at 260 nm) confirmed the ingredients after autoclaving and 3-year shelf-life are shown in FIG. 6.

TABLE 11

| | Δ Tan δ 1 Hz | |
|---|---|---|
| | After autoclaving | 45° C., 48 days |
| JUVEDERM ® Ultra Plus + | 0.059 | 0.020 |
| AA2G ™ 0.6% + Lidocaine 0.3% | | |
| JUVEDERM ® Ultra Plus + | 0.016 | 0.007 |
| AA2G ™ 0.6% + TPGS 1.5% + | | |
| lidocaine 0.3% | | |

### Example 12

### AA2G™ Promotes Collagen Synthesis

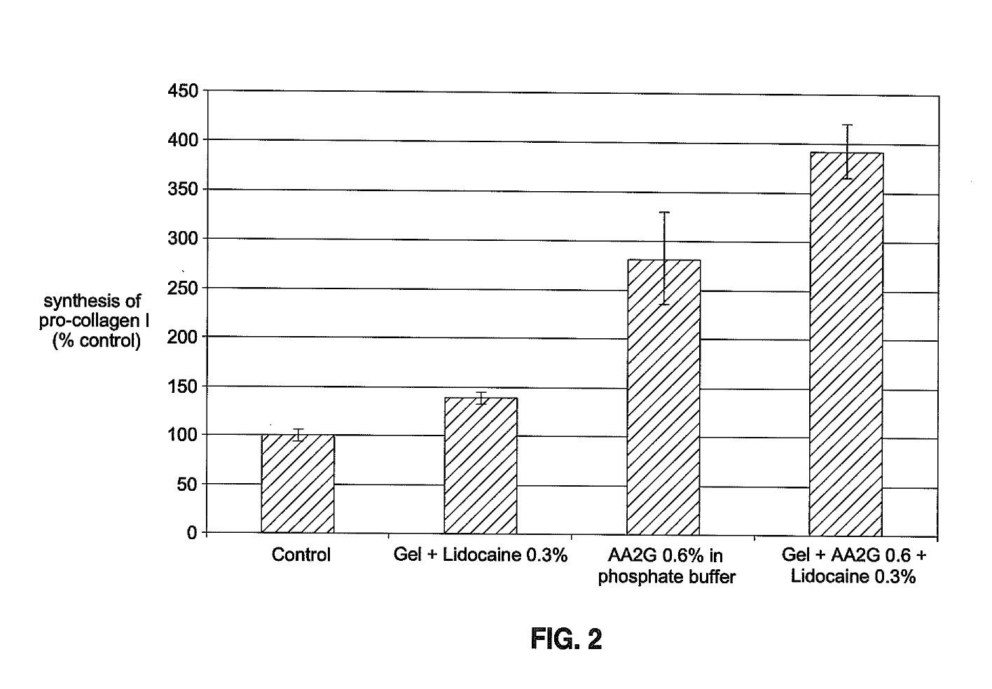

Human skin fibroblasts were cultured in a 12 wells plate. At confluence, 100 μL of each compound (Juverderm® FORMA with 0.3% lidocaine, Juverderm® FORMA+AA2G™ 0.6%+Lidocaine 0.3% and Phosphate Buffer with 0.6% AA2G) was deposited in a culture insert (porosity of 0.4 μm), which was itself laid on the fibroblast monolayers. In parallel, a control without treatment was performed. Cultures were incubated for 72 hours and each experimental condition was conducted done in triplicate. At the end of incubation, cell viability was verified by microscopic observation and MTT reduction assay. Pro-collagen I secretion was measured using ELISA kit. The presence of 0.6% AA2G™ in a hyaluronic acid gel containing 0.3% lidocaine increased pro-collagen synthesis by a factor 3 (+292%), whereas JUVEDERM® gel with 0.3% lidocaine showed an increase of 40% of the pro-collagen secretion (see FIG. 2).

### Example 13

### AA2G™ Protects NaHA from Oxidative Degradation

The effect of AA2G™ on NaHA oxidative degradation was studied. Oxidation testing was used as it allows testing of the resistance of a NaHA matrix to free radicals.

Degradation by free radicals was simulated on a rheometer (Haake Rheostress 600) by addition of 1/7 ratio of H₂O₂ 30% on the surface of a spread gel measured with a controlled stress rheometer according to the following method: frequency of 1 Hz with 0.8% controlled strain, during 3600 s at 35° C. The time value is taken at 5 Pa/s.

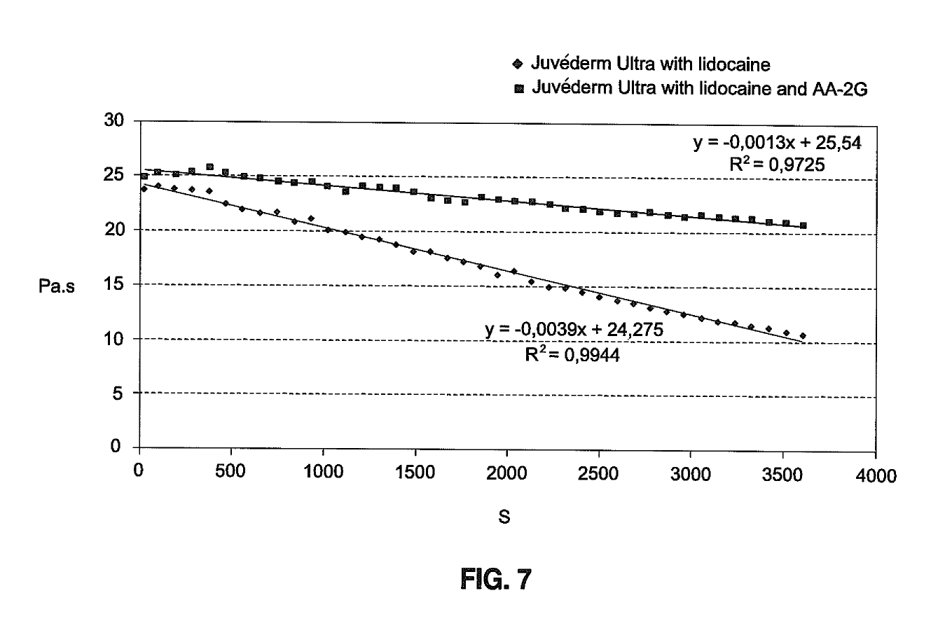

Further, a comparison of antioxidant properties for JUVEDERM® Ultra with AA2G™ 0.6%/Lidocaine 0.3% formulation (15 800 s) versus NaHA matrix JUVEDERM® Ultra with Lidocaine (4 942 s) showed that the gel containing AA2G™ and lidocaine is more stable with respect to free radical activity (see FIG. 7). AA2G™ protected against oxidative degradation by a factor of 3.

### Example 14

### Implantation Study

A gel containing AA2G™ at 0.6% (=6 mg/g=2.10⁻² mM) was implanted in the deep dermis and subcutaneous tissues in rats. Histological evaluation at 1 week showed some mononuclear cells (lymphocytes and plasmocytes) around the implants in all implantation sites (test and control). They were also associated with macrophages. The gel containing AA2G™ appeared to be less inflammatory. The irritation index in test samples (AA2G™+NaHA) was 9.9 compared to 12.3 in controls (NaHA only). Table 12 shows the histological results at 1 week, 1 month, and 3 months. The irritation score of AA2G™ gel are (for each implantation time) lower than control (gel without AA2G™)

TABLE 12

| | NAHA + AA2G + Lido |
|---|---|
| Biocompatibility ISO 10993 | |
| Cytotoxicity | ✓ (non cytotoxic) |
| Irritation | ✓ (non irritant) |
| Sensitization | ✓ (non sensitizing) |
| Implantation Test | |
| 1 week | ✓ (no skin reaction) |
| 3 weeks | ✓ (no skin reaction) |
| 3 months | ✓ (no skin reaction) |

### Example 15

### Incorporation of Dexpanthenol in NaHA Gel Formulations

Dexpanthenol was incorporated into a NaHA matrix JUVEDERM® Ultra Plus with Lidocaine (with 0.3% w/w lidocaine) with a content of 1% w/w. The gel was autoclaved. The gel obtained was clear and uncolored before and after autoclaving. The gel properties after autoclaving are shown in Table 13.

TABLE 13

| | After autoclaving | |
|---|---|---|
| | Extrusion | |
| Formulation | force (N) | Δ Tan δ 1 Hz |
| JUVEDERM ® Ultra Plus with | PASSED | 0.026 |
| Lidocaine (0.3%) + Dexpanthenol 1% | | |

### Example 16

### Effect of the Incorporation of Dexpanthenol in NAHA Gel Formulations

The shelf-life at 45° C. during 30 days was tested of the formulations prepared in example 15 and the NaHA matrix JUVEDERM® Ultra Plus XC. The gel was clear, uncolored. Rheology data of the gels containing dexpanthenol 1% w/w and lidocaine 0.3% w/w are shown in Table 14.

TABLE 14

| | After 30 days at 45° C. |
|---|---|
| Formulation | Δ Tan δ 1 Hz |
| JUVEDERM ® Ultra Plus with | −0.071 |
| Lidocaine (0.3%) + Dexpanthenol 1% | |

Rheological studies showed an increase of the stability of the gel in the presence of the additive.

### Example 17

### Incorporation of Epinephrine in NaHA Gel Formulations

Epinephrine was incorporated into a NaHA matrix (JUVEDERMφ Ultra Plus) with a 10 ppm epinephrine bitartrate. The gel was autoclaved. The gel obtained was clear and uncolored before and after autoclaving. The gel properties after autoclaving are shown in Table 15.

TABLE 15

After autoclaving

| | Extrusion | |
|---|---|---|
| Formulation | force (N) | Δ Tan δ 1 Hz |
| JUVEDERM ® Ultra Plus + | PASSED | 0.165 |
| epinephrine bitartrate 10 ppm | | |

A degradation of the gel was observed by rheological analysis.

### Example 18

### Incorporation of Epinephrine in NaHA Gel Formulations

Epinephrine was incorporated into a NaHA matrix (JUVEDERM® Ultra Plus) with 0.3% lidocaine and 10 ppm epinephrine bitartrate. The gel was autoclaved. The gel obtained was clear and colored after autoclaving. The gel properties after autoclaving are shown in Table 16.

TABLE 16

After autoclaving

| | Extrusion | |
|---|---|---|
| Formulation | force (N) | Δ Tan δ 1 Hz |
| JUVEDERM ® Ultra Plus + | PASSED | 0.092 |
| Lidocaine 0.3% + epinephrine | | |
| bitartrate 10 ppm | | |

A slight degradation of the gel was observed by rheological analysis.

### Example 19

### Effect of Additional Ingredient on the Stability of Gel Containing Epinephrine and Lidocaine

The shelf-life at 45° C. during 60 days was tested of the formulations prepared in example 18 and the NaHA matrix JUVEDERM® Ultra Plus. The gel was clear, slightly colored. Rheology data of the gels containing epinephrine bitartrate (10 ppm), lidocaine (0.3% w/w) is shown in Table 17.

TABLE 17

| | After 60 days at 45° C. |
|---|---|
| Formulation | Δ Tan δ 1 Hz |
| JUVEDERM ® Ultra Plus + | 0.185 |
| Lidocaine 0.3% + epinephrine | |
| bitartrate 10 ppm | |

After a stability of 60 days at 45° C., the gel containing epinephrine and lidocaine was unstable.

### Example 20

### Incorporation of Epinephrine in NaHA Gel Formulations Containing an Antioxidant

Epinephrine was incorporated into a NaHA matrix (JUVEDERM® Ultra Plus) with epinephrine bitartrate (10 ppm) and mannitol (0.9 or 4.5% w/w). The gels were autoclaved. The gel with 4.5% mannitol was clear and uncolored before and after autoclaving whereas with 0.9% mannitol was slightly colored. The gel properties after autoclaving is shown in Table 18.

TABLE 18

| | After autoclaving | |
|---|---|---|
| | Extrusion | |
| Formulation | force (N) | Δ Tan δ 1 Hz |
| JUVEDERM ® Ultra Plus + epinephrine | PASSED | 0.047 |
| bitartrate 10 ppm + mannitol 0.9% | | |
| JUVEDERM ® Ultra Plus + epinephrine | PASSED | 0.015 |
| bitartrate 10 ppm + mannitol 4.5% | | |

No degradation was observed for both gels.

### Example 21

### Effect of Additional Ingredient on the Stability of Gel Containing Epinephrine and an Antioxidant

The shelf-life at 45° C. during 60 days was tested of the formulations prepared in example 20 and the NaHA matrix JUVEDERM® Ultra Plus. The gels were clear, slightly colored. Rheology data of the gels containing epinephrine bitartrate (10 ppm) and mannitol (0.9 or 4.5% w/w) is shown in Table 19.

TABLE 19

| | After 60 days at 45° C. |
|---|---|
| Formulation | Δ Tan δ 1 Hz |
| JUVEDERM ® Ultra Plus + epinephrine | 0.061 |
| bitartrate 10 ppm + mannitol 0.9% | |
| JUVEDERM ® Ultra Plus + epinephrine | 0.006 |
| bitartrate 10 ppm + mannitol 4.5% | |

After a stability of days at 45° C., both gels containing epinephrine, lidocaine and manitol were stable. The composition containing 4.5% mannitol was more stable.

### Example 22

### Incorporation of Epinephrine in NaHA Gel Formulations Containing Lidocaine and Antioxidant

Epinephrine was incorporated into a NaHA matrix (JUVEDERM® Forma) with epinephrine bitartrate (20 ppm), lidocaine (0.3% w/w) and mannitol (4.5% w/w). The gel was autoclaved. The gel obtained was clear slightly colored after autoclaving. The gel properties after autoclaving are shown in Table 20.

TABLE 20

| | After autoclaving | |
|---|---|---|
| | Extrusion | |
| Formulation | force (N) | Δ Tan δ 1 Hz |
| JUVEDERM ® Forma + Lidocaine 0.3% + | PASSED | 0.026 |
| epinephrine bitartrate 20 ppm + | | |
| mannitol 4.5% | | |

No degradation was observed.

### Example 23

### Effect of Additional Ingredient on the Stability of Gel Containing Epinephrine, Lidocaine and an Antioxidant

The shelf-life at 45° C. during 60 days was tested of the formulations prepared in example 22 and the NaHA matrix JUVEDERM® Forma. The gel was clear, slightly colored. Rheology data of the gel containing epinephrine bitartrate (20 ppm), lidocaine (0.3% w/w) and mannitol (4.5% w/w) is shown in Table 21.

TABLE 21

| | After 60 days at 45° C. |
|---|---|
| Formulation | Δ Tan δ 1 Hz |
| JUVEDERM ® Forma + epinephrine | −0.030 |
| bitartrate 20 ppm + mannitol 4.5% | |

The gel is stable after 60 days at 45° C.

### Example 24

### Incorporation of Synephrine in NaHA Gel Formulations Containing Lidocaine and Antioxidant

Synephrine was incorporated into a NaHA matrix Juverderm Ultra Plus with Lidocaine (with 0.3% w/w lidocaine) with a content of 100 ppm of synephrine. The gel was autoclaved. The gel obtained was clear and uncolored before and after autoclaving. The gel properties after autoclaving is shown in Table 22.

TABLE 22

| | After autoclaving | |
|---|---|---|
| | Extrusion | |
| Formulation | force (N) | Δ Tan δ 1 Hz |
| JUVEDERM ® with lidocaine (0.3%) + | PASSED | −0.006 |
| synephrine 100 ppm | | |

### Example 25

### Effect of Additional Ingredient on the Stability of Gel Containing Synephrine and Lidocaine

The shelf-life at 45° C. during 60 days was tested of the formulations prepared in example 24 and the NaHA matrix JUVEDERM® Ultra Plus with Lidocaine. The gels was clear, uncolored. Rheology data of the gel containing synephrine 100 ppm and lidocaine 0.3% w/w is shown in Table 23.

TABLE 23

| | After 60 days at 45° C. |
|---|---|
| Formulation | Δ Tan δ 1 Hz |
| JUVEDERM ® Ultra Plus with | −0.028 |
| lidocaine (0.3%) + synephrine 100 ppm | |

Rheological studies showed an slightly increase of the stability of the gel in the presence of the additive.

### Example 26

### Incorporation of Phenylephrine in NaHA Gel Formulations Containing Lidocaine

Phenylephrine was incorporated into a matrix JUVEDERM® Ultra Plus with Lidocaine (with 0.3% w/w lidocaine) with a content of 100 ppm phenylephrine. The gel was autoclaved. The gel obtained was clear and uncolored before and after autoclaving. The gel properties after autoclaving are shown in Table 24.

TABLE 24

| | After autoclaving | |
|---|---|---|
| | Extrusion | |
| Formulation | force (N) | Δ Tan δ 1 Hz |
| JUVEDERM ® Ultra Plus with Lidocaine | PASSED | −0.002 |
| 0.3% + Phenylephrine 100 ppm | | |

### Example 27

### Effect of Additional Ingredient on the Stability of Gel Containing Phenylephrine and Lidocaine

The shelf-life at 45° C. during 60 days was tested of the formulations prepared in example 26 and the NaHA matrix JUVEDERM® Ultra Plus with Lidocaine. The gel was clear, uncolored. Rheology data of the gel containing phenylephrine 100 ppm and lidocaine 0.3% w/w are shown in Table 25.

TABLE 25

| | After 60 days at 45° C. |
|---|---|
| Formulation | Δ Tan δ 1 Hz |
| JUVEDERM ® Ultra Plus with Lidocaine | −0.017 |
| (0.3%) + Phenylephrine 100 ppm | |

Rheological studies showed a slight increase of the stability of the gel in the presence of the additive.

### Example 28

### Incorporation of Naphazoline in NaHA Gel Formulations Containing Lidocaine and Antioxidant

Naphazoline was incorporated into a matrix Juverderm Ultra Plus with Lidocaine (with 0.3% w/w lidocaine) with a content of 100 ppm. The gel was autoclaved. The gel obtained was clear and uncolored before and after autoclaving. The gel properties after autoclaving are shown in Table 26.

TABLE 26

| | After autoclaving | |
|---|---|---|
| | Extrusion | |
| Formulation | force (N) | Δ Tan δ 1 Hz |
| JUVEDERM ® Ultra Plus with Lidocaine | PASSED | −0.003 |
| (0.3%) + Naphazoline 100 ppm | | |

### Example 29

### Effect of Additional Ingredient on the Stability of Gel Containing Naphazoline and lidocaine

The shelf-life at 45° C. over 60 days was tested of the formulations prepared in example 28 and the NaHA matrix JUVEDERM® Ultra Plus with Lidocaine. The gel was clear, uncolored. Rheology data of the gel containing naphazoline 100 ppm and lidocaine 0.3% w/w is shown in Table 27.

TABLE 27

| | After 60 days at 45° C. |
|---|---|
| Formulation | Δ Tan δ 1 Hz |
| JUVEDERM ® Ultra Plus with Lidocaine | −0.008 |
| 0.3% + Naphazoline 100 ppm | |

### Example 28

### Treatment Example

A woman, age 37, presents with fine lines around her eyes and deeper wrinkles on the sides of her mouth. She receives injections of a formulation of Example 10. She receives the injections in the fine lines and in the wrinkles once a week for 3 weeks and notices a visible improvement in the appearance of her skin.

### Example 29

### Alternate Treatment Example

A 59 year old man presents with wrinkles between his eyebrows and in the nasolabial folds. He receives injections of the formulation of Example 11, every 3 months. A visible improvement in the wrinkles is seen.

### Example 28

### Alternate Treatment Example

A 35 year old woman presents with fine lines across her forehead. She receives injections of the formulation of Example 12, once a week for two weeks, and notices an improvement in the appearance of the skin on her forehead.

### Example 30

### Alternate Treatment Example

A woman, age 44, presents with uneven texture on her right cheek resulting from a loss of collagen due to aging. She receives injections of the formulation of Example 13, in her cheek to build up the areas where the collagen has been lost. A visible improvement is seen in the texture of the skin on her cheek after 3 series of injections over a 2 week period of time.

### Example 31

### Alternate Treatment Example

A 35 year old man presents with a deep wrinkle across his chin and fine lines on the sides of his eyes. He receives the formulation of Example 14 along the sides of his eyes. He receives 2 series of injections in his chin, spaced 1 week apart. The fine fines and wrinkle are visibly diminished after treatment.

### Example 32

### Alternate Treatment Example

A woman, age 62, presents with wrinkles across her forehead, on the sides of her eyes, in the nasolabial folds, and a scar on her chin. She receives injections of the formula of Example 15 each week for one month. After the injections, the appearance of the wrinkles and the scar is visibly diminished.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in fight of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

What is claimed is:

1. A dermal filler formulation comprising hyaluronic acid (HA) and at least one additional constituent selected from the group consisting of vitamin B, C and vitamin E, wherein the formulation exhibits greater stability than an HA gel formulation without the additional constituent.

2. The formulation of claim 1, wherein the HA is cross-linked.

3. The formulation of claim 1, wherein the HA is present in an amount of about 1 to about 60 mg/mL.

4. The formulation of claim 1, wherein the HA is present in an amount of about 10 to about 40 mg/mL.

5. The formulation of claim 1, wherein the vitamin C is ascorbyl-2-glucoside.

6. The formulation of claim 5, wherein the ascorbyl-2-glucoside is AA2G™.

7. The formulation of claim 1, wherein the vitamin E is TPGS.

8. The formulation of claim 1, further comprising epinephrine.

9. The formulation of claim 1, wherein the gel is monophasic.

10. The formulation of claim 1, wherein the formulation is stable for at least 2 years.

11. The formulation of claim 1, wherein the formulation is stable for at least 3 years.

12. The formulation of claim 1, wherein the formula is stable after sterilization by a process selected from steam sterilization, filtration, gamma radiation, or microfiltration.

13. The formulation of claim 1, further comprising an anesthetic.

14. The formulation of claim 13, wherein the anesthetic is selected from the group consisting of lidocaine, benzocaine, butamben, dibucaine, oxybuprocaine, pramoxine, proparacaine, proxymetacaine, and tetracaine.

15. The formulation of claim 1, wherein the formulation is suitable for injection.

16. The formulation of claim 1, wherein the additional constituent is present in an amount of about 0.1% to about 3% w/w.

17. The formulation of claim 1, wherein the additional constituent provides the formulation with improved rheological properties resulting in less extrusion force required for administration compared to an HA gel formulation without the additional constituent.

18. The formulation of claim 1, wherein the additional constituent is added directly to the HA gel.

19. The formulation of claim 1, wherein the additional constituent is incorporated into the HA gel in a liposome, micelle, or polymerized vesicle.

20. A method of treating fine lines, wrinkles, fibroblast depletions, or scars afflicting a subject comprising administering to the subject an effective amount of a formulation comprising HA and at least one additional constituent selected from the group consisting of vitamin B, C and vitamin E, wherein the formulation exhibits greater stability than an HA gel formulation without the additional constituent, and wherein the appearance of the fine lines, wrinkles, fibroblast depletions, or scars is diminished.

21. The method of claim 20, wherein the formulation is injected into the facial skin of the subject.

22. The method of claim 20, wherein the additional constituent is present in an amount of about 0.1% to about 3% w/w.

23. The method of claim 20, wherein the formulation further comprises at least one selected from the group consisting of epinephrine, lidocaine, benzocaine, butamben, dibucaine, oxybuprocaine, pramoxine, proparacaine, proxymetacaine, tetracaine, and a combination thereof.

24. The method of claim 20, wherein the additional constituent provides the formulation with improved rheological properties resulting in less extrusion force required for administration compared to an HA gel formulation without the additional constituent.

25. The method of claim 20, wherein the HA is cross-linked.

26. The method of claim 20, wherein the HA is present in an amount of about 1 to about 60 mg/mL.

27. The method of claim 20, wherein the vitamin C is ascorbyl-2-glucoside.

28. The method of claim 20, wherein the vitamin E is TPGS.

29. The method of claim 27, wherein the ascorbyl-2-glucoside is AA2G™.

30. A dermal filler comprising about 1 to about 60 mg/mL HA and an additional constituent selected from the group of ascorbyl-2-glucoside and TPGS, wherein the dermal filler exhibits greater stability than a dermal filler comprising HA without the additional constituent.

31. A dermal filler comprising at least 90 wt % high molecular weight HA, about 0 to about 10 wt % of a low molecular weight HA, a cross linker, and about 0.1 wt % to about 1wt % of an ascorbyl-2-glucoside.

32. The dermal filler of claim 31, wherein the HA is about 4% to about 11% crosslinked.

33. The dermal filler of claim 31, wherein the crosslinker is 4-butane diol diglycidyl ether (BDDE).

34. The dermal filler of claim 31, wherein the HA is present at a concentration of about 15 mg to about 24 mg/mL dermal filler.
